(19) United States
(12) Patent Application Publication
(10) Pub. No.: US 2026/0265661 A1
(43) Pub. Date: Sep. 10, 2026

(54) BIOREACTOR APPARATUS

(71) Applicant: VUALA LTD, London (GB)

(72) Inventors: Yiu Wa Ma, London (GB); Hon Lam Ma, London (GB)

(21) Appl. No.: 19/491,320

(22) PCT Filed: Jun. 13, 2024
(86) PCT No.: PCT/GB2024/051516

§ 371 Date: Dec. 8, 2025

(30) Foreign Application Priority Data
Jun. 13, 2023 (GB) 2308837.0

(51) Int. Cl. C12M 3/04; B09B 3/60; B09B 101/70; C12M 1/00; C12M 1/36

(52) U.S. Cl. CPC C12M 27/10; B09B 3/60; C12M 27/20; C12M 29/06; C12M 41/48; B09B 2101/70

(57) ABSTRACT

The present disclosure relates to disposal of food waste. The present disclosure provides a bioreactor apparatus for converting food waste to a slurry, the apparatus comprising: a drum having: a drum surface defining an interior volume, wherein the interior volume is configured to receive food waste; a plurality of holes in the drum surface to permit fluid to enter and exit the interior volume; and, a central longitudinal axis, wherein the drum is configured to rotate about the central longitudinal axis; and, one or more sprayers configured to spray fluid at the drum to thereby permit fluid sprayed at the drum to enter the interior volume of the drum.

### FIELD OF THE INVENTION

The present disclosure relates to disposal of food waste, in particular, to a bioreactor apparatus for disposal of waste, for example, organic waste such as food waste, parts of a bioreactor apparatus, and a method of disposing of waste.

### BACKGROUND

HK1224249 describes an organic garbage automatic classification system that efficiently processes kitchen garbage. The system includes a grinding separator, consisting of a cylinder body, an rotating agitating device set inside the cylinder body. The rotating agitating device grinds the organic waste into organic particles.

HK30014313 describes a system comprises a cylinder body for receiving waste, a stirring device rotatably arranged in the cylinder body which grinds the waste to produce organic thick liquid using a grinding separator.

The rotating agitating device in HK1224249 and the stirring device in HK30014313 can become jammed when solid components introduced to the system (e.g. cutlery or bones). This can result in downtime for the systems and/or costly maintenance and repair of the systems.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

An aspect of the disclosure provides a bioreactor apparatus for converting food waste to a slurry, the apparatus comprising: a drum having: a drum surface defining an interior volume, wherein the interior volume is configured to receive food waste; a plurality of holes in the drum surface to permit fluid to enter and exit the interior volume; and, a central longitudinal axis, wherein the drum is configured to rotate about the central longitudinal axis; and, one or more sprayers configured to spray fluid at the drum to thereby permit fluid sprayed at the drum to enter the interior volume of the drum.

Typical systems comprise a stirrer which rotates inside a cylindrical body to agitate or break down waste therein (e.g. see background section above). The stirrer can jam or become damaged when solid waste become trapped between said stirrer and the cylindrical body. This can result in downtime for the systems and/or costly maintenance and repair of the typical systems. Embodiments described herein do not require a stirrer to break down waste, instead, the drum rotates to thereby move and break down waste disposed therein. Therefore, advantageously, embodiments may avoid the downtime and costly maintenance associated with typical systems.

The drum may be a cylindrical drum (e.g. a drum with a cylindrical shape). In such examples, the cylindrical drum comprises a cylindrical surface defining an interior volume. The cylindrical drum may be configured to rotate about a central longitudinal axis of the cylindrical drum. The central longitudinal axis may refer to a straight line passing through the centre of each circular cross-section of the cylinder.

The drum may be a conical drum (e.g. a drum with a conical shape or with a frustoconical shape). In such examples, the conical drum comprises a conical surface or frustoconical surface defining an interior volume. The conical drum may be configured to rotate about a central longitudinal axis of the conical drum. The central longitudinal axis may refer to a straight line passing through the centre of each circular cross-section of the cone or conical frustum (e.g. a right conical frustum).

The drum may comprise one or more baffles on an interior of the drum surface, wherein the one or more baffles are configured to mechanically break solid parts of food waste disposed within the interior volume.

The baffles may further contribute to movement and break down of waste in the drum (e.g. in addition to that due to the rotation of the drum) which may improve the efficacy of the bioreactor apparatus. Furthermore, the baffles may not be susceptible to jamming unlike the typical systems.

Each of one or more baffles may comprise a plurality of teeth. For example, said teeth may have one or more sharp edges configured to penetrate and/or cut waste impacted by said teeth. For example, the teeth may pierce or cut packaging (e.g. plastic food packaging) into smaller pieces.

The one or more sprayers may be disposed adjacent to the drum. For example, when the bioreactor apparatus is configured for use, the sprayers may be disposed above the drum.

Typical systems comprise two sets of sprayers, which, when these systems are configured for use, are arranged with one set above a cylindrical chamber and the other below said chamber. In such examples, the set above the chamber is for introducing water to the chamber and the set below is for spraying water at drainage holes in the chamber to keep these drainage holes clear from debris (e.g. so that they are still able to drain fluid from the chamber). In these typical systems, the set of sprays below the chamber (in particular) may be responsible for a great deal of water use. In examples described herein a single set of sprayers may be provided (e.g. above the drum); because the whole drum rotates (e.g. around the central longitudinal axis), the plurality of holes therein are kept clear due to gravity (debris covering said holes may fall away when these holes are inverted relative to the ground in use). Advantageously when a single set of sprayers is provided a reduced amount of fluid (e.g. mains water) may be required compared to typical systems which comprise two sets of sprayers.

The bioreactor apparatus may comprise a slurry tank configured to collect fluid which exits from the drum, wherein the slurry tank is disposed under the drum. The slurry tank may be removable. Advantageously a removable slurry tank may enable simple and relatively quick removal of slurry from the bioreactor apparatus which reduces bioreactor apparatus down time. Typical bioreactors require drainage of a non-removable tank which may lead to comparatively longer bioreactor down time. Embodiments may not require down time for removal of the slurry tank and so advantageously eliminate down time for removal of slurry.

The drum may comprise a drum port having: an open configuration wherein solid waste is discharged from the drum via the drum port; and, a closed configuration wherein solid waste is not discharged from the drum via the drum port; the bioreactor apparatus further comprising: a solid waste tank configured to collect solid waste discharge from the drum via the openable drum port. The drum port may permit food waste to be added to the drum.

The solid waste tank may be removable. A removable solid waste tank may enable simple and relatively quick removal of solid waste from the bioreactor apparatus which reduces bioreactor apparatus down time.

The slurry tank may be disposed closer to the first longitudinal end than to the second longitudinal end; the solid waste tank is disposed closer to the second longitudinal end than to the first longitudinal end; the drum port is disposed closer to the second longitudinal end than to the first longitudinal end and above at least a portion of the solid waste tank.

The drum port may be provided by: an opening in a portion of the drum suitable for solid waste to pass therethrough; and, a sheathe member disposed around the exterior of at least a portion of the drum; wherein: the sheathe cylinder is either: rotatable around the longitudinal axis and relative to the drum; or movable along the longitudinal axis and relative to the drum; the drum port is in the closed configuration when the sheathe cylinder covers the opening; and, the drum port is in the open configuration when the sheathe cylinder does not cover the opening.

In examples comprising a cylindrical drum, the sheathe member may be a sheathe cylinder. In such examples, the sheathe cylinder may be movable relative to the cylindrical drum. For example, the cylindrical cylinder may be rotatable around the longitudinal axis (e.g. the same axis as the cylindrical drum) and/or movable longitudinally along the central longitudinal axis.

In examples comprising a conical drum, the sheathe member may be a conical frustum. In such examples, the sheathe member may be movable relative to the conical drum. For example, the sheathe member may be rotatable around the longitudinal axis (e.g. the same axis as the conical drum).

In typical systems which have a relatively complicated arrangement, comprising a vibrating grate is provided to separate slurry and solid pieces of waste. Embodiments of the disclosure comprising a drum port which permit selective removal of solid waste from the drum. For example, these embodiments may not require a vibrating grate, which comparatively reduces the size of the bioreactor apparatus, simplifies its manufacture and maintenance, and the power required to operate it.

The one or more baffles may be configured to: move solid parts of food waste towards the first longitudinal end when the drum is rotated in a first rotational direction around the longitudinal axis; and, move solid parts of food waste towards the second longitudinal end when the drum is rotated in a second rotational direction around the longitudinal axis.

The drum may comprise one or more baffles which are shaped to: move the solid waste to a first longitudinal end when the drum is rotated in the first rotational direction; and, to move the solid waste to a second longitudinal end when the drum is rotated in the second rotational direction. For example, the baffles may have any suitable shape described herein, such as a helix shape.

Rotating the drum in the second rotational direction may switch the drum port from the closed configuration to the open configuration. For example, the sheathe member may be rotatable relative to the drum through a fixed angular displacement equal to or greater than its own angular extent around the longitudinal axis. The sheathe member may be freely relatable relative to the drum.

Rotation of the drum in the first rotational direction may cause the drum to rotate relative to the sheathe member until the sheathe member engages the drum wherein the relative position of the sheathe member and the drum leaves the opening in the drum covered by the sheathe member i.e. the drum port is in the closed configuration.

Rotation of the drum in the second rotational direction may cause the drum to rotate relative to the sheathe member until the sheathe member engages the drum wherein the relative position of the sheathe member and the drum leaves the opening in the drum uncovered by the sheathe member i.e. the drum port is in the open configuration.

In other words, advantageously a passive means of switching the drum port between an open and closed configuration may be provided which may reduce power requirements of the bioreactor apparatus.

Alternatively, the switching of the drum port between an open and closed configuration may be performed using an actuator controller by a controller of the bioreactor apparatus e.g. a motor.

In examples the solid waste may be removed from the drum via the drum port at a preset time interval. For example, the controller may be configured to rotate the drum to move solid waste to a given longitudinal end of the drum and then the controller may be configured to open the drum port.

Typical systems such as those described in the background section above may require manual discharge (e.g. by a maintenance worker) of solid waste from bioreactors. Therefore, advantageously, the embodiments described herein may provide a bioreactor apparatus which may automatically remove solid waste from the drum. Typical systems may require manual sorting of solid waste (e.g. undigestible waste) from food waste (e.g. waste which will be digested by the bioreactor apparatus) by a worker before the waste is disposed within such a system. Therefore, advantageously, the embodiments described herein may provide a bioreactor apparatus which may automatically sort solid waste from food waste in the drum, removing the need for a worker to do this.

An alternative method of removing waste from the drum is set out herein. In such examples, a sheathe member is disposed over the opening of the drum port and the sheathe member is connected to the drum with one or more elastic members (e.g. springs) and an actuator is provided configured to push the sheathe member against the one or more elastic members thereby exposing the opening. An open configuration is provided when the actuator is retreats to pull the sheathe member along the longitudinal axis against the elastic member(s) to thereby expose the opening. A closed configuration is provided when the actuator extends and the elastic member(s) push the sheathe member along the longitudinal axis to cover the opening.

The bioreactor apparatus may comprise a slurry recirculation system configured to recirculate slurry from the slurry tank to the interior of the drum. In examples, the slurry recirculation system may be in fluid communication with one or more sprayers (e.g. either the same one or more sprayers for spraying water from the water mains to the drum or another one or more sprayer, for example, a sprayer specifically for spraying slurry into the drum. Advantageously, the slurry recirculation system may provide slurry which is comparatively more concentrated (i.e. than when no slurry recirculation system is provided) which may reduce the volume of slurry produced per unit mass of food waste (e.g. the volume of slurry is less per unit mass of food waste when a slurry recirculation system is provided). Advantageously, slurry collection frequency may be reduced and accordingly logistics costs may accordingly be reduced.

Optionally, the apparatus may comprise a heater configured to heat water which is to be sprayed from one or more of the sprayers. Optionally, the slurry recirculation system may comprise a heater configured to heat fluid recirculated from the slurry to the one or more sprayers to a selected temperature. Optionally and in addition or alternatively, the slurry recirculation system may comprise a heater configured to heat slurry in the slurry recirculation system. Optionally, a heater may be provided in, at, and/or proximal to the slurry tank wherein the heater is configured to heat slurry in the slurry tank. The selected temperature may be a temperature of 30° C. or a temperature above 30° C., for example, 35° C. or more preferably 37° C. For example, the fluid may comprise a culture of microorganisms (e.g. any combination of bacteria, enzymes, and fungi) configured to breakdown (e.g. using biological and chemical processes) waste disposed in the drum. In examples, a mixed culture of microorganisms may comprise specialised microorganisms (e.g. microorganisms configured to breakdown food waste). The specialised microorganisms, may comprise any of: lactobacillus; prevotella; saccharomyces cerevisiae; and, ruminococcin. Advantageously, the specialised microorganisms may be configured to rapidly breakdown of food waste solids (e.g. compared to other, non-specialised microorganisms). The specialised microorganisms may be added to the drum with selected media wherein the selected media is configured to cultivate the specialised microorganisms. The selected temperature may be selected to improve the efficacy of these microorganisms, for example, a temperature may be or close to an optimum temperature of said microorganisms (e.g. 37° C.).

The bioreactor apparatus may comprise an air-tight housing configured to enclose, within an interior of the housing, the drum, one or more sprayers, and the slurry tank, wherein the air-tight housing is configured to prevent odorous gas (e.g. odour-causing matter such as odour-causing chemicals) exiting the interior of the housing.

Advantageously, the air-tight housing may prevent odour causing elements (e.g. any of solids, liquids and gases) from leaving the housing which may prevent unpleasant odours. Typical systems comprise vacuum blowers and/or chemical odour removal units to control odour emission which requires power and chemicals (which are consumable) whereas the present disclosure may provide an air-tight housing which is a passive measure. Furthermore, the vacuum blowers may be relatively loud whereas an air-tight housing does not generate a noise. The comparative manufacture and maintenance cost of the air-tight housing of the present disclosure may be lower than the vacuum blowers of typical systems.

Herein an air-tight housing refers to a housing wherein at least one of the following conditions apply:
The housing comprises a plurality of panels and the edges wherein the panels meet are sealed;
The food waste input is in a closed configuration during operation (e.g. rotation of the drum);
The drum port is in a closed configuration during operation (e.g. rotation of the drum);
The slurry tank is provided in a separate compartment to thereby confine smell from the slurry tank to said separate compartment.

The bioreactor apparatus may comprise: a food waste input having: a first configuration wherein the food waste input is open to receive food wase and closes off the interior of the housing; and, a second configuration wherein the food waste input is closed to receive food and opens the food waste input to the interior of the housing to permit the food waste in the food waste input to enter the interior of the drum.

Advantageously, the food waste input has two configurations which provide a physical barrier between the interior of the housing (including, for example, the drum) and an exterior in both the first configuration and the second configuration which may have any of the following advantages. An advantage may be that odour causing particles are not permitted to leave the interior of the housing via the food waste input (or, very low amounts may escape via this route). An advantage may be that direct access to the drum may not be possible through the food waste input in either the first or second configuration, which may prevent injury, if for example, a user put their arm within housing and it was injured by the rotation of the drum.

The food waste input may be disposed above the second longitudinal end of the drum to permit food to enter the interior of the drum via the drum port. Advantageously, food input to the bioreactor apparatus via the food waste input may enter the drum via the drum port (also disposed at the second longitudinal end of the drum), which may provide a simplified design.

The bioreactor apparatus may comprise: a caddy food waste loader configured to automatically input food waste in a food caddy into the food waste inlet; and/or, a bin food waste loader configured to automatically input food waste in a food bin into the food waste inlet.

Any loader may be provided as an additional module to the bioreactor apparatus itself. For example, the bioreactor apparatus may be configured so that one loader may be replaced by another loader e.g. by removal of the manual food waste loader and replacement with a caddy food waste loader or bin food waste loader, or vice versa. Conveniently, a single bioreactor apparatus may be produced which is useable in a variety of situations without (e.g. manual loading of waste into the food waste input) or with a loader (e.g. caddy or bin loader).

An aspect provides a method of operating a bioreactor apparatus, the method comprising: rotating a drum around a central longitudinal axis wherein the drum comprising a plurality of holes disposed through a radial surface thereof; and, spraying liquid from a one or more sprayers at the drum.

The liquid may comprise any of: water; a microorganism culture; and, components of food waste (e.g. solid particulates and/or dissolved components).

Typical methods of operation comprise rotating a stirrer inside a body to agitate or break down waste therein (e.g. see background section above). The stirrer can jam or become damaged when solid waste become trapped between said stirrer and the body. This can result in downtime for the systems and/or costly maintenance and repair of the typical systems. Embodiments described herein do not require a stirrer to break down waste, instead, the method comprises rotating a drum to thereby move and break down waste disposed therein. Therefore, advantageously, embodiments may avoid the downtime and costly maintenance associated with typical systems.

An aspect provides a method of operating a bioreactor apparatus the method comprising: rotating a drum in a first direction around a longitudinal axis wherein the drum comprising a plurality of holes disposed through a radial surface thereof, to thereby move solid parts of food waste towards the second longitudinal end; and, rotating the drum in a second direction around the longitudinal axis to thereby move solid parts of food waste towards the second longitudinal end.

Typical methods of operation (e.g. such as methods of operation of the systems described in the background section above) may require manual discharge (e.g. by a maintenance worker) of solid waste from bioreactors. Therefore, advantageously, the embodiments described herein may provide a method of automatically removing solid waste from the drum.

Methods describe herein may comprise a step of: introducing a culture of microorganisms in the drum wherein the microorganisms are configured to break down the food waste.

Typical systems do not comprise provide methods of operating bioreactors which includes a step of providing microorganisms. Advantageously, microorganisms may improve, and/or accelerate the rate of, break down of food waste to slurry.

The methods described herein may be performed using any bioreactor apparatus described herein.

An aspect provides a computer readable non-transitory storage medium comprising a program for a computer configured to cause a processor to perform any method described herein.

### BRIEF DESCRIPTION OF THE DRAWINGS

Embodiments of the disclosure will now be described, by way of example only, with reference to the accompanying drawings, in which:

FIG. 1 illustrates a lateral cross-sectional plan view of a bioreactor apparatus;

FIG. 2 illustrates a lateral plan view of a cylindrical drum of the bioreactor apparatus;

FIG. 3 illustrates a longitudinal cross-sectional plan view of the cylindrical drum of FIG. 2;

FIG. 4A illustrates a lateral plan view of a conical drum having a frustoconical shape;

FIG. 4B illustrates a lateral plan view of a conical drum having a conical shape;

FIG. 5 illustrates a flowchart depicting a method of operating a bioreactor apparatus;

FIG. 6 illustrates a flowchart depicting a method of operating a bioreactor apparatus;

FIG. 7A illustrates a lateral cross-sectional plan view of a bioreactor apparatus;

FIG. 7B illustrates a lateral plan view of a cylindrical drum of the bioreactor apparatus; FIG. 7C illustrates a longitudinal cross-sectional plan view of the cylindrical drum of FIG. 7B;

FIG. 8A illustrates a simplified plan view of a drum with a sheathe member arranged in a closed configuration;

FIG. 8B illustrates a plan view of a drum with a sheathe member arranged in an open configuration.

In the drawings, like reference signs indicate like elements.

### SPECIFIC DESCRIPTION

Provided herein is a bioreactor apparatus for breaking down food waste into a slurry. Any components of the food waste which cannot be broken down by the bioreactor apparatus are separated from the slurry by the bioreactor apparatus. The bioreactor apparatus is configured for use with a culture of microorganisms which aid the break down of food waste into a slurry.

The term breaking down or break down herein refers to the transformation of one or more inlet material into one or more output materials. For example, the inlet material is food waste and output material is a slurry.

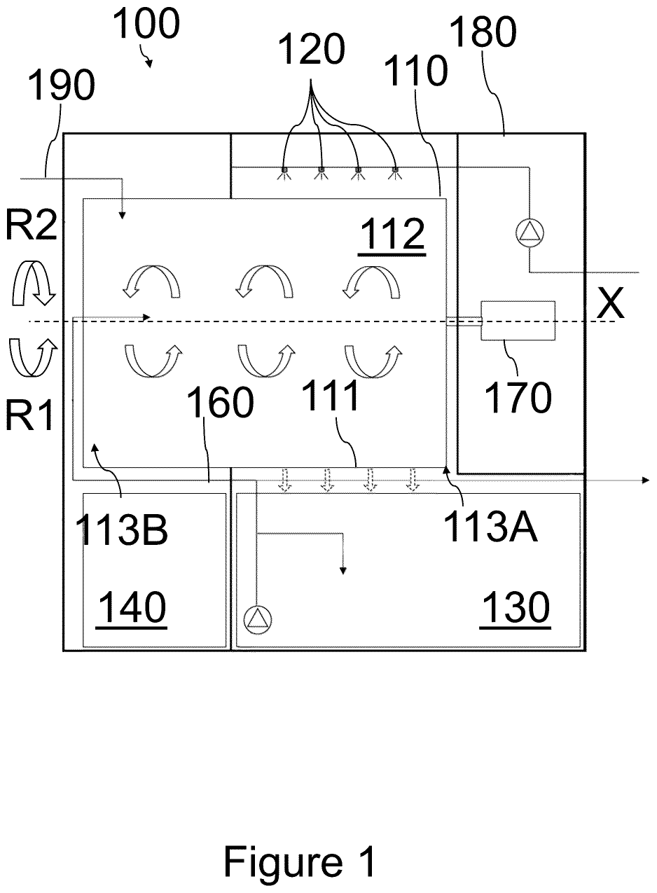

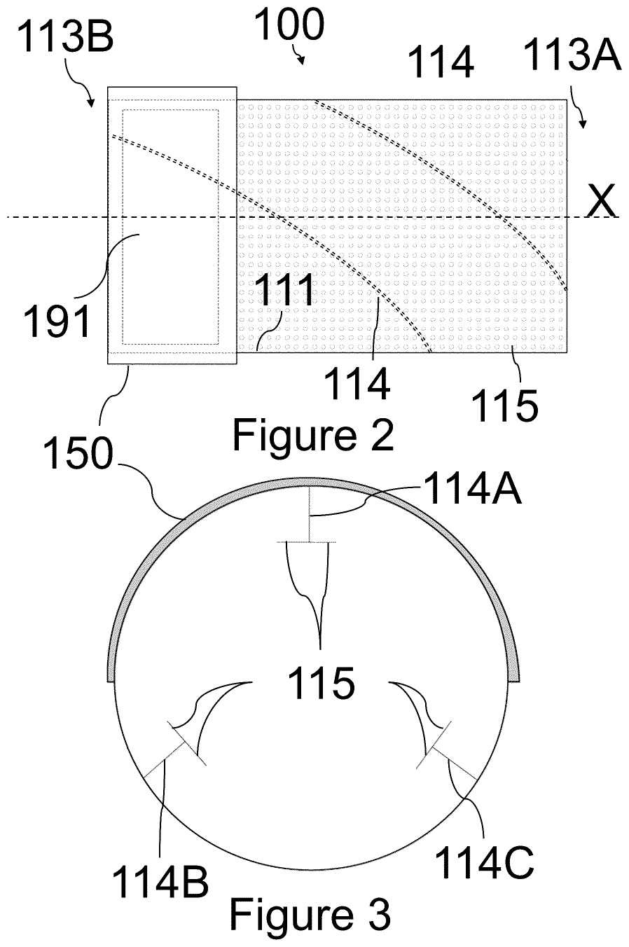

FIG. 1 illustrates a lateral cross-sectional plan view of a bioreactor apparatus 100; FIG. 2 illustrates a lateral plan view of a cylindrical drum 110 of the bioreactor apparatus 100; FIG. 3 illustrates a longitudinal plan view of the cylindrical drum 110 of FIG. 2.

The bioreactor apparatus comprises: a cylindrical drum 110; a plurality of sprayers 120; a slurry tank 130; a solid waste tank 140; a sheathe cylinder 150 (not shown in FIG. 1 for simplicity, but shown in FIGS. 2 to 4); a slurry recirculation system 160; a controller 170; an air-tight housing 180; a food waste inlet 190.

In examples, the cylindrical drum 110 may be replaced with a conical drum (e.g. a drum having a conical shape or a frustoconical shape).

The air-tight housing 180 defines an interior 181. The air-tight housing 180 encloses the cylindrical drum 110, the plurality of sprayers 120, the slurry tank 130 the solid waste tank 140, the sheathe cylinder 150 (e.g. these elements are disposed within the interior of the air-tight housing 180). The air-tight housing 180 encloses at least part of the slurry recirculation system 160, and optionally, the controller 170 (in FIG. 1 the controller 170 is disposed within the air-tight housing 180 but may disposed outside the air-tight housing 180 in other examples).

The sheathe cylinder is an example of a sheathe member. In examples wherein the drum is a conical drum the sheathe member is a sheathe member which is a conical frustum. The cylindrical drum 110 comprises: a cylindrical surface 111 (e.g. a drum surface); an interior volume 112 defined by the cylindrical surface 111; a central longitudinal axis X about which the cylindrical drum 110 is rotatable; a first longitudinal end 113A; and a second longitudinal end 113B; an opening 191 in a portion of the cylindrical drum 110 which forms a part of the food waste outlet 190. There are a plurality of holes 116 disposed in the cylindrical surface 111.

The cylindrical drum 110 is configured to rotate about the rotational axis X. The controller 170 is configured to control the rotation of the cylindrical drum 110 e.g. the controller comprises a motor coupled to the cylindrical drum, wherein rotation of the motor provides rotation of the cylindrical drum 110. The cylindrical drum is configured to rotate in both directions R1 & R2. Rotation of the cylindrical drum 110 is configured to move waste and fluid disposed within the interior 112. The movement of the waste and fluid in the interior 112 results in collisions between any combination of: the elements of the waste and the fluid; one element of waste with another element of waste; and, the waste and the cylindrical surface 111 of the cylindrical drum 110. All collisions may contribute to breaking the waste into smaller parts to thereby generate a slurry.

The cylindrical drum 110 comprises a plurality of baffles 114. Each baffle is disposed within the interior volume 112 of the cylindrical drum 111 and is attached to the cylindrical surface 111. In the present example, three baffles are provided, namely, a first baffle 114A, a second baffle 114B, and a third baffle 114C. The plurality of baffles are disposed equidistantly around the longitudinal axis X. Each baffle 114 has a helical shape, centred on the rotational axis X of the cylindrical drum 110. For any given longitudinal cross section of the cylindrical drum 110, each baffle 114 is perpendicular to the cylindrical surface 111 (see FIG. 3). It will be appreciated that in examples, the baffles may be oblique to the cylindrical surface.

Each baffle 114 comprises a plurality of teeth 115. In the present example, the teeth 115 of a given baffle 114 are arranged perpendicular to that baffle 114. These teeth are configured to hold fluid in the region between the teeth, baffle, and cylindrical surface during rotation of the cylindrical drum 110 which may improve the transformation of the waste into slurry. The teeth may have an edge configured to penetrate the waste (e.g. the edge has a dimension parallel to the radius of the cylindrical drum 110 so that at typical rotational speeds it may penetrate, cut, or shear the waste) which may improve the transformation of waste into slurry.

Each baffle is connected to an interior surface (i.e. the cylindrical surface 111 of the cylindrical drum 110). In examples, one or more baffles may be unitarily formed with cylindrical surface 111 of the cylindrical drum 110 i.e. they may be a single piece of material.

The baffles 114 are shaped so that solid waste can be driven to the second longitudinal end 113B of the cylindrical drum 110. The helically-shaped baffles 114 when rotated in the second rotational direction, drive solid waste to second longitudinal end 113B. In the present example, the baffle shape is a helix, but any baffle with a shape which, when rotated continuously in a single direction has a non-time reversible geometry (i.e. the baffles do not look the same irrespective of the direction of rotation of the cylindrical drum) may be used to provide the same effect. Another example of a suitably-shaped baffle is a helical baffle with a varying pitch e.g. the pitch along the longitudinal direction is not constant.

The cylindrical drum 110 is configured to drive solid waste to the second longitudinal end 113B of the cylindrical drum 110. The opening 191 and the cylindrical sheathe are disposed at the second longitudinal end 113B arranged over the solid waste tank 140 (also disposed at the second longitudinal end 113B). The longitudinal extent of the opening 182 is less than or equal to the longitudinal extent of the solid waste tank 140, which thereby prevents the solid waste missing the solid waste tank 140 (and, for example, entering the slurry tank 130).

The plurality of sprayers 120 are configured to spray fluid at the cylindrical drum 110. The plurality of sprayers 120 are configured to receive fluid from a water main (e.g. a municipal water supply). The sprayers 120 are disposed adjacent to the cylindrical drum 110 (e.g. vertically above the cylindrical drum 110 in use). The fluid sprayed from the sprayers 120 at the cylindrical drum 110. Each hole 116 provides fluid communication between the interior volume 112 of the cylindrical drum and the outside of the cylindrical drum. The holes 116 permit fluid (e.g. water) sprayed at the cylindrical drum 110 to enter the cylindrical drum 110.

In examples, a single sprayer may be provided. Any spray provided may comprise a single orifice or a plurality of orifices from which fluid may be sprayed.

The plurality of holes 116 are configured to permit slurry generated in the interior 112 of the cylindrical drum 110 to exit the interior 112. The slurry may exit the cylindrical drum continuously during operation (e.g. when the cylindrical drum is rotated) or when rotation of the cylindrical drum 110 is ceased.

The slurry tank 130 is configured to catch slurry exiting the interior 112 of the cylindrical drum 110 e.g. in use, the slurry tank 130 is disposed below the cylindrical drum 110. The slurry tank is removable e.g. removeable from the air-tight housing 180. The slurry tank 130 may comprise a slurry level sensor (described in more detail herein).

The slurry recirculation system 160 is configured to collect fluid (e.g. slurry) which drains from the cylindrical drum 110 (e.g. via the plurality of holes 116). The slurry recirculation system 160 comprises a fluid mover (e.g. a pump) to move the slurry to the interior of the drum. The slurry recirculation system 160 may be moved into the interior of the drum via the opening 191 of the drum port. For example, the drum may intermittently stop to receive, via the opening, slurry from the slurry recirculation system 160 to increase the number of microorganisms in the drum. For examples, the slurry recirculation system may comprise one or more sprayers (distinct from sprayers 120) to spray slurry into the interior of the drum. In other examples, a hole disposed on the longitudinal axis of the drum to permit continuous recirculation of fluid to the drum may be provided. The slurry recirculation system 160 is configured to heat the fluid (e.g. using a heater, not shown in the drawings) to a selected temperature. In examples, the selected temperature may improve breakdown of the organic matter, for example, the temperature is selected to improve the efficacy of the chemical processes which the microorganisms use to break down the organic matter.

The controller 170 is configured to control the rotation of the cylindrical drum 110 (e.g. the speed, direction, duration etc.). The controller 170 is configured to control the slurry recirculation system (e.g. control the fluid mover, such as a pump; control heating of the fluid, for example, by controlling a heater thereof). If a slurry level sensor is provided, then the controller is configured to receive an indication therefrom.

In examples with a slurry level sensor, the slurry level sensor may provide a signal when the tank is full so as to initiate slurry collection (e.g. a signal may be sent to a slurry collection centre, via the controller 170, to inform the centre that collection is required). The signal may also prevent any further food waste input. The system will continue to operate (e.g. including drum rotation and recirculation of slurry).

The slurry level sensor may also generate a signal when the slurry tank is close to empty to thereby provide a protection when pumping out slurry from the tank to suction truck. For example, the slurry level sensor may generate a signal which causes an alarm (e.g. audio and/or visual indication) that the slurry tank is close to empty to alert an operator of a pump emptying the tank to cease pumping. Advantageously, a portion of microorganisms may be left in the tank which may prevent addition of a new culture, and/or damage to the slurry tank and/or collection pump may be prevented.

In examples, in response to the indication, the controller 170 may be configured to cease operation of the bioreactor apparatus (e.g. prevent food waste being introduced into the cylindrical drum via the food waste input and/or cease rotation of the cylindrical drum 110 and/or cease fluid exiting the sprayers 120). In such examples, in response to the indication, the controller 170 is configured to alert a user that the slurry tank is full (e.g. visually and/or audibly). In other examples, the slurry level sensor may be self-contained and may alert a user itself.

The food waste inlet 190 is operated to provide food waste (or any other organic waste, for example, garden waste) to the interior 112 of the cylindrical drum 110. Operation of the food waste inlet 190 is described herein. The food waste passes through the opening 191 in the portion of the cylindrical drum 110 thereby entering the interior 112 of the cylindrical drum 110.

The sheathe cylinder 192 is rotated relative to the cylindrical drum 110 to thereby close off the opening 191.

The cylindrical drum 110 is rotated about the rotational axis X. The cylindrical drum may be rotated in a single direction around the rotational axis X (e.g. either R1 or R2) or in both directions in sequence (e.g. R1 then R2). The rotational speed (e.g. the angular rotational speed) may be varied which may advantageously improve mechanical breakdown of food waste (e.g. in comparison to using a single rotational speed).

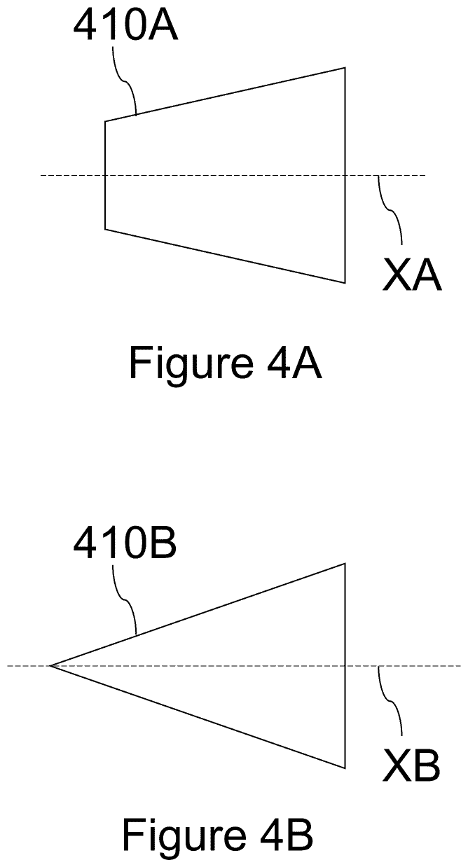

In examples, the drum may be a conical drum. Conical drums may be either conically shaped or frustoconically shaped. FIG. 4A illustrates a lateral plan view of a conical drum 410A having a frustoconical shape. The conical drum 410A is rotatable around a central longitudinal axis XA. FIG. 4B illustrates a lateral plan view of a conical drum 410B having a conical shape. The conical drum 410B is rotatable around a central longitudinal axis XB.

The sprayers 120 spray fluid at the cylindrical drum 110. When the bioreactor apparatus 100 is arranged for use, the sprayers 120 are arranged above the cylindrical drum 110.

The rotation of the cylindrical drum 110 causes movement of the organic matter and fluid disposed within the interior 112. Movement of the organic matter and fluid in the interior 112 results in collisions between the elements of the organic matter and the fluid, fluid and the organic matter, and the organic matter and the cylindrical surface of the cylindrical drum 110, which all contribute to breaking the organic matter into smaller parts which form part of the slurry.

The baffles 114 and teeth 115 which may have sharp edges to cut into waste. The baffles 114 and teeth 115 of the cylindrical drum impact waste comprising organic matter and inorganic matter in the interior. For example, inorganic matter in the interior (e.g. food packaging) may be broken into smaller pieces by the baffles 114 and teeth 115.

A microorganism culture is provided in the fluid which is sprayed into the interior 112. The microorganism culture is configured to biologically break down the organic matter. Specifically the microorganism culture chemically converts components of the organic matter from one or more inlet components to one or more output components wherein at least one of the inlet components is chemically distinct from at least one of the output components. The output components form part of the slurry.

By the action of the rotation of the cylindrical drum 110 and the chemically processes due to the microorganism culture the organic matter in the cylindrical drum 110 is converted to a slurry.

Slurry in the interior 112 of the cylindrical drum 110 passes through the plurality of holes 116 in the cylindrical surface 111 e.g. the slurry egresses from the interior 112 under gravity. Slurry passes through the holes 116 irrespective of whether the cylindrical drum 110 is rotated i.e. slurry exits the drum when the drum rotates and when the drum is stationary.

The slurry exiting the interior 112 of the cylindrical drum 110 enters a slurry tank 130. When the bioreactor apparatus is arranged for use, the slurry tank 130 is arranged below the cylindrical drum 110.

The slurry recirculation system 160 collects fluid (e.g. slurry) which drains from the cylindrical drum 110 (e.g. via the plurality of holes 116). This collected fluid is recirculated using a fluid mover (e.g. a pump) to reintroduce the fluid to the interior 112 of the cylindrical drum 110 as described herein. Furthermore, the heater of the slurry recirculation system 160 (not shown in the drawings) heats the fluid to a selected temperature. As described herein, the temperature is selected to improve breakdown of the organic matter, for example, the temperature is selected to improve the efficacy of the chemical processes which the microorganisms use to break down the organic matter.

The slurry collects in the slurry tank 130. The slurry tank 130 may comprise a slurry level sensor (not shown in the drawings) which provides an indication when slurry tank reaches a selected level (e.g. a level indicating that the slurry tank 130 is filled to capacity). The slurry level sensor may comprise a float configured to provide the indication when the float is raised vertically by an increase in the level of the slurry. The indication may be sent to the controller 170 and the controller 170 may cease operation of the bioreactor apparatus 100 in response to the indication. The controller 170 may provide an alert to alert a user to the amount of slurry in the slurry tank 130. Accordingly the user may cease operation of the bioreactor apparatus and/or remove the slurry tank 130.

The slurry tank 130 is removable. For example, a slurry tank 130 filled to capacity can be removed, thereby removing the slurry from the bioreactor apparatus 100 and then subsequently another slurry tank (e.g. identical to the removed slurry tank, but empty) may be disposed within the bioreactor apparatus 100. The removable slurry tank 130 enables simple and relatively quick removal of slurry from the bioreactor apparatus 100 which reduces bioreactor apparatus 100 down time. Advantageously a waste removal truck with suction apparatus may not be required to empty slurry from the apparatus. In examples, the bioreactor apparatus 100 may continue operation (e.g. rotation of the drum) whilst the slurry tank is removed, thereby foregoing the need for any down time. Typical bioreactors require drainage of a non-removable tank which leads to comparatively longer bioreactor apparatus down time. In some example, the slurry tank may not be removable and instead may be pumped empty e.g. by a waste removal truck.

Solid waste (e.g. matter which cannot be reduced to slurry, for example, food packaging and bones) is removed from the interior 112 of the cylindrical drum 110 and moved to the solid waste tank 140. By rotating the cylindrical drum 110 around axis X in a given direction the baffles 114 drive the solid waste to above the solid waste tank 140. In the present example, the cylindrical drum is rotated in the second rotational direction R2 which causes the baffles 114 to drive to solid waste to the second longitudinal end 113B of the cylindrical drum 110 and, therefore, above the solid waste tank 140. The solid waste may be considered disposed above the solid waste tank 140 after a predetermined number of revolutions of the cylindrical drum 110.

When the solid waste is disposed above the solid waste tank 140, the sheathe cylinder 150 is rotated relative to the cylindrical drum 110 to expose the opening 191 in the cylindrical drum 110 which results in the solid waste falling through the opening under gravity into the solid waste tank 140. As described herein, the longitudinal extent of the opening 191 is less than or equal to the longitudinal extent of the solid waste tank 140, which thereby prevents the solid waste missing the solid waste tank 140.

The solid waste tank 140 is removable. For example, a solid waste tank 140 filled to capacity can be removed, thereby removing the solid waste from the bioreactor apparatus 100 and then subsequently another solid waste tank (e.g. identical to the removed solid waste tank, but empty) may be disposed within the bioreactor apparatus 100. Alternatively, the solid waste tank may be drained The removable solid waste tank 140 enables simple and relatively quick removal of solid waste from the bioreactor apparatus 100 which reduces bioreactor apparatus 100 down time. In examples, the solid waste tank may be a rigid receptacle (e.g. a plastic or metal tub) or the solid waste tank may be a flexible receptacle (e.g. a plastic bag).

The bioreactor apparatus may be configured to include any of a manual food waste loader, a caddy food waste loader and a bin food waste loader. The food waste is provided to the food waste inlet 190 by any of the manual food waste loader, a caddy food waste loader and a bin food waste loader i.e. each of the manual food waste loader, a caddy food waste loader and a bin food waste loader may provide food waste to the food waste inlet 190.

The three aforementioned loaders may be provided as additional modules to the bioreactor apparatus itself and the bioreactor apparatus may be configured so that one loader may be replaced by another loader e.g. by removal of the manual food waste loader and replacement with a caddy food waste loader or bin food waste loader, or vice versa. Conveniently, a single bioreactor apparatus may be produced which is useable in a variety of situations by simply providing a suitable inlet unit.

The food waste input has two configurations between which is can move: a first configuration wherein the food waste input is open to receive food waste and closes off the interior of the housing; and, a second configuration wherein the food waste input is closed to receive food and opens the food waste input to the interior of the housing to permit the food waste in the food waste input to enter the interior of the cylindrical drum.

The food waste input 190 is provided by a hollow prism having an obtuse segment longitudinal cross section which is rotatable about a central longitudinal axis. An open part of the segment (e.g. between the edges of the obtuse segment) provide an opening which permits food waste to be received by and removed from the food waste input 190. A closed part (e.g. a solid part of the obtuse segment) is arranged to close off openings in the housing.

In the first configuration the open part exposes the opening to the exterior of the housing and the closed part closes off the interior of the housing. In the second configuration the open part exposes the opening to the interior of the housing and the closed part closes off the exterior of the housing.

The bioreactor apparatus is arranged in a state ready to receive food waste when the drum port is arranged so that the opening in the cylindrical drum faces the food waste input. The drum port is in the closed configuration (e.g. the sheathe cylinder 150 is disposed over the opening in the cylindrical drum 110) and the food waste input 190 is in a second configuration.

Next the drum port is arranged in the closed configuration and the food waste input 190 is arranged in the first configuration. Next the drum port is arranged in the open configuration and the food waste input 190 is arranged in the first configuration. Next food waste is inserted into the food waste inlet. Next the drum port is arranged in the open configuration and the food waste input 190 is arranged in the second configuration wherein the food waste exits the food waste inlet and enters the cylindrical drum via the opening.

Finally the drum port is arranged in the closed configuration and the food waste input 190 is arranged in the second configuration. Now operation of the bioreactor apparatus may resume.

The bioreactor apparatus may comprise: a caddy food waste loader configured to automatically input food waste in a food caddy into the food waste inlet; or, a bin food waste loader configured to automatically input food waste in a food bin into the food waste inlet.

Any loader may be provided as an additional module to the bioreactor apparatus itself. For example, the bioreactor apparatus may be configured so that one loader may be replaced by another loader e.g. by removal of the manual food waste loader and replacement with a caddy food waste loader or bin food waste loader, or vice versa. Conveniently, a single bioreactor apparatus may be produced which is useable in a variety of situations without (e.g. manual loading of waste into the food waste input) or with a loader (e.g. caddy or bin loader).

Occasionally, the food waste input is cleaned. To clean the food waste input, water is sprayed through one or more sprayers (e.g. either the sprayers 120 or through one or more separate sprayers) to thereby flush residues of food waste therefrom.

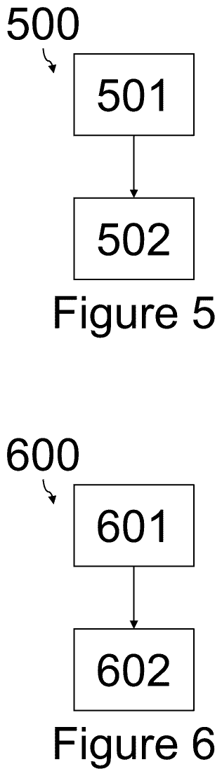

FIG. 5 illustrates a flowchart depicting a method 500 of operating a bioreactor apparatus. The method comprises the steps of:
Rotating, 501, a cylindrical drum around the longitudinal axis wherein the cylindrical drum comprising a plurality of holes disposed through a radial surface thereof; and,
Spraying, 502, liquid from a one or more sprayers at the cylindrical drum.

Method 500 may comprise an additional step of: introducing a culture of microorganisms in the cylindrical drum wherein the microorganisms are configured to break down the food waste.

FIG. 6 illustrates a flowchart depicting a method 600 of operating a bioreactor apparatus. The method comprises the steps of:

Rotating, 601, a cylindrical drum in a first direction around a longitudinal axis wherein the cylindrical drum comprising a plurality of holes disposed through a radial surface thereof, to thereby move solid parts of food waste towards the first longitudinal end; and,

Rotating, 602, the cylindrical drum in a second direction around the longitudinal axis to thereby move solid parts of food waste towards the second longitudinal end.

Method 600 may comprise an additional step of: introducing a culture of microorganisms in the cylindrical drum wherein the microorganisms are configured to break down the food waste.

The one or more sprayers are configured to spray fluid wherein said fluid is water. In examples, the fluid may consist essentially of water.

The fluid may comprise water and the microorganisms are configured to transform organic matter (e.g. food waste) into a slurry. The slurry comprises particulate organic matter (e.g. small particles of the original organic matter), transformed organic matter (e.g. organic matter which is different from the original organic matter, for example, because it has undergone a chemical reaction).

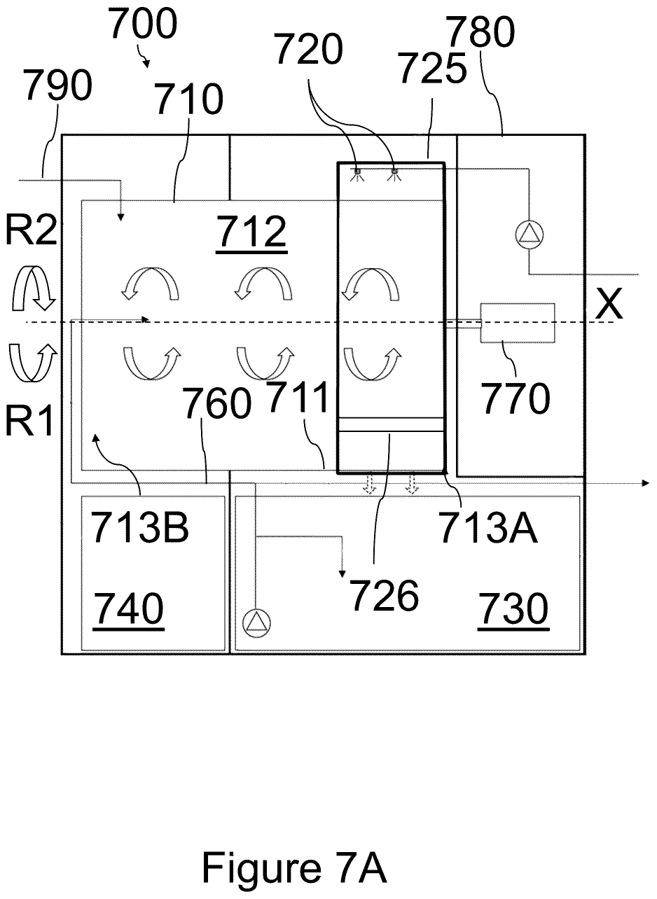

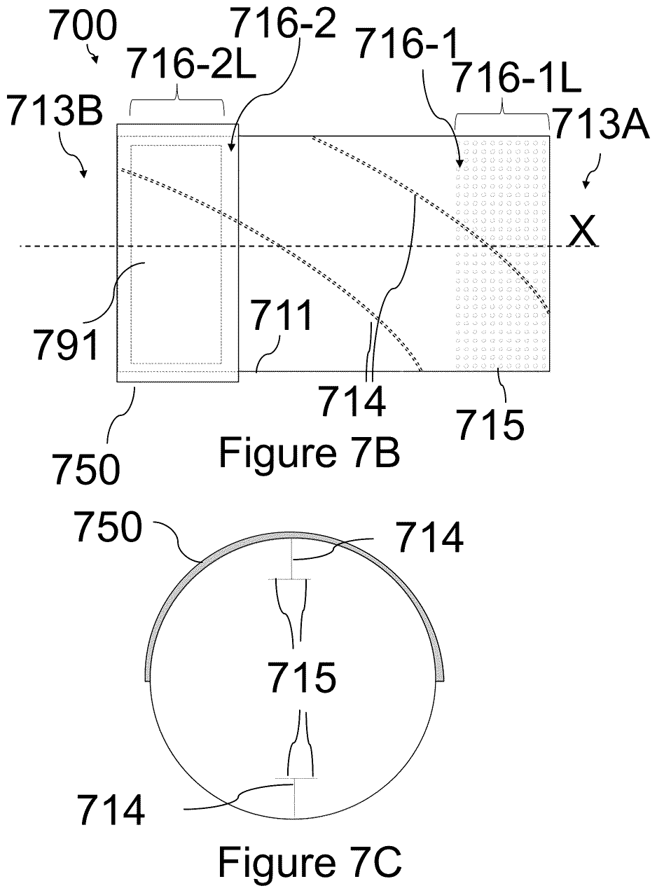

FIG. 7A to 7C illustrate an example bioreactor apparatus;

The bioreactor apparatus 700 comprises: a drum 710; a plurality of sprayers 720; an outer casing 725; scrapers 726; a slurry tank 730; a solid waste tank 740; a slurry recirculation system 760; a controller 770; an air-tight housing 780; and a food waste inlet 790.

As shown in FIG. 7A, the drum 710 is cylindrical. It will be appreciated that the drum may have a conical shape (e.g. a drum having a conical shape or a frustoconical shape).

The air-tight housing 780 contains the other components of the bioreactor apparatus in the manner as the air-tight housing 180 shown in FIG. 1.

The food waste inlet 790 is used to input and output waste into the drum. The food waste inlet 790 comprises an opening 791 of the drum, a sheathe member 750, and actuators 792, 793 all described in more detail below.

The drum 710 comprises: a cylindrical surface 711 (e.g. a drum surface); an interior volume 712 defined by the cylindrical surface 711; a central longitudinal axis X about which the drum 710 is rotatable (e.g. the central longitudinal axis X is the longitudinal axis of the cylindrical surface 711); a first longitudinal end 713A; and a second longitudinal end 713B; an opening 791 in a portion of the drum 710 which forms a part of the opening 791. There are a plurality of holes 716 disposed in the cylindrical surface 711.

The sheathe member 750 is any member configured to close off (e.g. block) the opening 791. The sheathe member 750 in FIGS. 7A to 7D is a portion of a cylinder, in this particular, example, the sheathe member 750 is an arcuate portion of a cylinder. The sheathe member 750 has an arcuate angular extent equal to or greater than the arcuate angular extent of the food waste inlet e.g. it has an arc length equal to over greater than) the arcuate angular extent of the opening 791.

The plurality of holes 716 are disposed in a longitudinal portion of the cylindrical surface 711. In particular, the cylindrical surface 711 has two longitudinal portions (although the cylindrical surface 711 may be a unitary member), a first longitudinal portion 716-1 having a first longitudinal length 716-1L and a second longitudinal portion 716-2 having a second longitudinal length 716-2L.

The first longitudinal portion 716-1 is a portion of the cylindrical surface 711 comprising the first longitudinal end 713A of the drum 710. The second longitudinal portion 716-2 is a portion of the cylindrical surface 711 comprising the second longitudinal end 713B of the drum 710. When the bioreactor 700 is configured for use, the first longitudinal portion 716-1 is disposed above the slurry tank 730 and the second longitudinal end portion 716-2 is disposed above the solid waste tank 740. The first longitudinal length 716-1L is equal to (or in examples, less than) a longitudinal length of the slurry tank 730 and the second longitudinal length 716-2L is equal to (or in examples, less than) a longitudinal length of the solid waste tank 740. The holes 716 are disposed in the first longitudinal portion 716-1 whereas none of the holes 716 are disposed in the second longitudinal portion 716-2. The opening 791 is disposed in the second longitudinal portion 716-2.

By providing a first longitudinal length 716-1L which is equal to or less than the longitudinal length of the slurry tank 730 advantageously permits slurry to exit the holes 716 to the slurry tank 730 and prevents slurry exiting the drum elsewhere (e.g. into the solid waste tank 740). Similarly, by providing a second longitudinal length 716-2L which is equal to or less than the longitudinal length of the solid waste tank 740 advantageously permits solid to food waste inlet 790 to the solid waste tank 740 and prevents solid waste exiting the drum elsewhere (e.g. into the slurry tank 730).

It will be appreciated that the sum of the first longitudinal length 716-1L and the second longitudinal length 716-2L may be less than the longitudinal length of the cylindrical surface 711.

In the present example, the first longitudinal portion 716-1 of the drum 710 is disposed entirely within the outer casing 725.

The cylindrical drum 710 is configured to rotate about the rotational axis X. The controller 770 is configured to control the rotation of the drum 710 e.g. the controller comprises a motor coupled to the drum, wherein rotation of the motor provides rotation of the drum 710 around the rotational axis X. The drum 710 is configured to rotate in both directions. Rotation of the drum 710 is configured to move waste and fluid disposed within the interior 712. The movement of the waste and fluid in the interior 712 results in collisions between any combination of: the elements of the waste and the fluid; one element of waste with another element of waste; and, the waste and the cylindrical surface 711 of the cylindrical drum 710. All collisions may contribute to breaking the waste into smaller parts to thereby generate a slurry.

The drum 710 comprises a plurality of baffles 714. Each baffle is disposed within the interior volume 712 of the cylindrical drum 711 and is attached to the cylindrical surface 711. Each baffle is connected to an interior surface (i.e. the cylindrical surface 711 of the drum 710). In examples, one or more baffles may be unitarily formed with cylindrical surface 711 of the cylindrical drum 710 i.e. they may be a single piece of material.

In the present example, two baffles are provided, namely, a first baffle 714A, and a second baffle 714B. It will be appreciated that any number of baffles may be provided, such as one baffle (which may advantageously simplify construction of the drum), thee baffles, four baffles, or any other number thereof. The plurality of baffles are disposed equidistantly around the longitudinal axis X. Each baffle 714 has a helical shape, centred on the rotational axis X of the drum 710. The baffles may be oblique or perpendicular to the cylindrical surface e.g. extending from the cylindrical surface toward the longitudinal axis X.

Each baffle 714 comprises a plurality of teeth 715. The teeth 715 of a given baffle 714 may be arranged to protrude away from the baffle in any direction from the baffle, for example, protrude along a radius of the drum (e.g. toward the longitudinal axis X) and/or protrude in a direction transverse to the radius of the drum. The teeth configured to penetrate waste in the drum e.g. the tooth may have a spiked portion or a cutting edge so upon collision (e.g. due to movement of the drum) between the tooth and waste the tooth penetrates and/or cuts the waste. In examples, the teeth may be configured to hold fluid in the region between the teeth, baffle, and cylindrical surface during rotation of the drum 710 which may improve the transformation of the waste into slurry.

In examples the teeth 715 may be arranged in the manner of a saw blade (e.g. a line of said teeth). The teeth may be arranged along the top of each baffle e.g. the surface of the baffle furthest from the surface which connects the baffle to the drum.

The baffles 714 are shaped so that solid waste can be driven to the second longitudinal end 713B of the cylindrical drum 710. The helically-shaped baffles 714 when rotated in the second rotational direction R2, drive solid waste to the second longitudinal end 713B. In the present example, the baffle shape is a helix, but any baffle with a shape which, when rotated continuously in a single direction has a non-time reversible geometry (i.e. the baffles do not look the same irrespective of the direction of rotation of the drum) may be used to provide the same effect. Another example of a suitably-shaped baffle is a helical baffle with a varying pitch e.g. the pitch along the longitudinal direction is not constant.

The drum 710 is configured to drive solid waste to the second longitudinal end 713B thereof e.g. when rotated in the second rotational direction. The opening 791 and the sheathe member 750 are disposed at the second longitudinal end 713B arranged over the solid waste tank 740 which is also disposed at the second longitudinal end 713B. The longitudinal extent of the opening 791 is less than or equal to the longitudinal extent of the solid waste tank 740, which thereby prevents the solid waste missing the solid waste tank 740 (and, for example, entering the slurry tank 730).

The plurality of sprayers 720 are configured to spray fluid at the drum 710. The plurality of sprayers 720 are configured to receive fluid from a water main (e.g. a municipal water supply). The sprayers 720 are disposed adjacent to the drum 710 (e.g. vertically above the drum 710 when the bioreactor 700 is arranged for use). Each hole 716 provides fluid communication between the interior volume 712 of the drum 710 and the outside of the cylindrical drum. The holes 716 permit fluid (e.g. water) sprayed at the drum 710 to enter the cylindrical drum 710.

In the present example, the sprayers 720 are disposed on the interior of the outer casing 725. In examples, a single sprayer may be provided. Any sprayer provided may comprise a single orifice or a plurality of orifices from which fluid may be sprayed.

In the example shown in FIG. 7A, the sprayers 720 are arranged to spray fluid at the first longitudinal portion 716-1 of the drum 710. In particular, the first longitudinal portion 716-1 comprises the plurality of holes 716. Given that the second longitudinal portion 716-2 of the drum 710 does not comprise any of the holes 716, none of the sprayer(s) are required to be arranged to direct fluid onto this portion of the drum.

The plurality of holes 716 are configured to permit slurry generated in the interior 712 of the drum 710 to exit the interior 712. The slurry may exit the drum continuously during operation (e.g. when the cylindrical drum is rotated) or when rotation of the drum is ceased. The slurry tank 730 is configured to catch slurry exiting the interior 712 via holes 716 e.g. in use, the slurry tank 730 is disposed below the first longitudinal portion 716-1 of the cylindrical drum 710. The slurry tank 730 is removable e.g. removeable from the air-tight housing 780. The slurry tank 730 may comprise a slurry level sensor (described in more detail herein).

One or more scrapers 726 may be provided. A scraper is configured to brush slurry off an outer surface of the drum 710. In more detail a scraper 726 may be arranged to contact a portion of the outer surface of the drum 710 so that slurry is brushed from the outer surface of the drum, and for example, falls into slurry tank 730 disposed below the scraper and/or drum. The scraper(s) 726 are connected to the outer casing 725.

In the present example, each of the one or more scrapers 726 may have a length parallel to the longitudinal axis X equal to or greater than the first longitudinal portion length 716-1L. That is, the scraper(s) may extend along the portion of the outer surface of the drum which comprises the plurality of holes 716 thereby to brush off slurry which egresses from the interior of the drum. In a preferred embodiment, two of

[symbol]

A slurry recirculation system 760 may be provided with the bioreactor 700 as set out above with reference to example shown in FIG. 1.

The controller 770 is configured to control the rotation of the drum 710 (e.g. the speed, direction, duration etc.). The controller 770 is configured to control the slurry recirculation system (e.g. control the fluid mover, such as a pump; control heating of the fluid, for example, by controlling a heater thereof). If a slurry level sensor is provided, then the controller is configured to receive an indication therefrom. These elements and their functions may be the same as that described in more detail above with reference to FIG. 1.

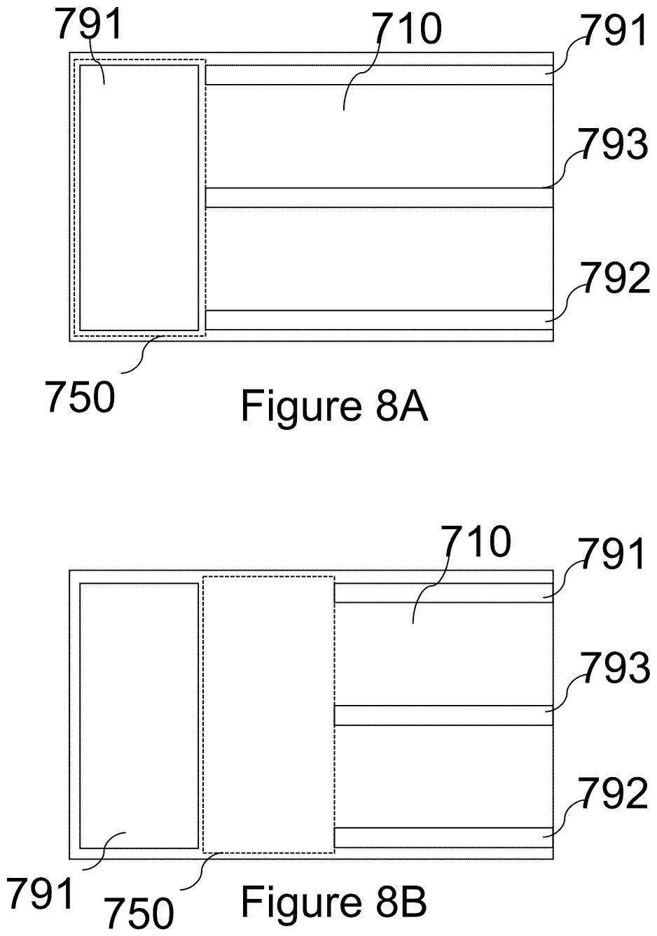

Operation food waste inlet 790 is described with reference to FIGS. 8A to 8F is described herein. The food waste inlet 790 is operable to permit waste to enter and exit the drum and to prevent waste from entering and exiting the drum.

Food waste passes through the opening 791 in the second longitudinal portion 716-2 of the drum thereby entering the interior 712 of the drum 710. Likewise, waste passes through the opening 791 in the second longitudinal portion 716-2 of the drum thereby exiting the interior 712 of the drum 710.

The sheathe member 750 is moved parallel to the longitudinal axis X relative to the drum 710. The sheathe member 750 is movable between a closed position and an open position. The closed position of the food waste inlet 790/the sheathe member 750 is shown in FIG. 8A. In the closed position the sheathe member 750 closes off (e.g. blocks) the opening 791. In the closed position ingress or egress of waste via the opening is prevented by the sheathe member 750.

The open position of the food waste inlet 790/the sheathe member 750 is shown in FIG. 8B. In the open position the sheathe member 750 exposes (e.g. does not block) the opening 791. In the open position ingress or egress of waste via the opening is permitted by the sheathe member 750.

In the present example, gas struts 792 are provided which are configured to move the sheathe member 750 to the closed position. One or more actuators may be provided to move the sheathe member to the open position. In the present example, a linear actuator 793 is provided to move the sheathe member to the open position. It will be appreciated that in some examples, a single actuator may be provided to provide both opening and closing functions.

Operation of the bioreactor 700 will now be described. The drum 710 is rotated about the rotational axis X. The cylindrical drum may be rotated in a single direction around the rotational axis X or in both directions in sequence. The rotational speed (e.g. the angular rotational speed) may be varied which may advantageously improve mechanical breakdown of food waste (e.g. in comparison to using a single rotational speed).

The sprayers 720 spray fluid at the cylindrical drum 110. When the bioreactor apparatus 700 is arranged for use, the sprayers 720 are arranged above the drum 710.

The rotation of the drum 710 causes movement of the organic matter and fluid disposed within the interior 712. Movement of the organic matter and fluid in the interior 712 results in collisions between the elements of the organic matter and the fluid, fluid and the organic matter, and the organic matter and the cylindrical surface of the cylindrical drum 710, which all contribute to breaking the organic matter into smaller parts which form part of the slurry.

The baffles 714 and teeth thereon may have sharp edges to cut into waste. The baffles 714 and teeth of the cylindrical drum impact waste comprising organic matter and inorganic matter in the interior. For example, inorganic matter in the interior (e.g. food packaging) may be broken into smaller pieces by the baffles 714 and teeth.

As set out elsewhere herein a microorganism culture is provided in the fluid which is sprayed into the interior of the bioreactor.

By the action of the rotation of the drum and optionally the chemically processes due to the microorganism culture the organic matter in the drum is converted to a slurry.

Slurry in the interior 712 of the drum 710 passes through the plurality of holes 716 in the first longitudinal portion 716-1 of the cylindrical surface 711 e.g. the slurry egresses from the interior 712 under gravity. Slurry passes through the holes 716 irrespective of whether the drum 710 is rotated i.e. slurry exits the drum when the drum rotates and when the drum is stationary.

The slurry exiting the interior 712 of the drum 710 enters a slurry tank 730. Example slurry tanks are described in more detail herein. It will be appreciated that when the bioreactor apparatus 700 is arranged for use, the slurry tank 730 is arranged below the drum 710.

The slurry recirculation system collects fluid (e.g. slurry) which drains from the drum 710 (e.g. via the plurality of holes 116) and recirculates it. The slurry recirculation system is described in more detail herein.

The slurry tank 730 may comprise a slurry level sensor the function of which is described in more detail herein.

Solid waste (e.g. matter which cannot be reduced to slurry, for example, food packaging and bones) is removed from the interior 712 of the cylindrical drum 710 to the solid waste tank 740. As set out herein, rotation of the drum 710 around axis X in a given direction the baffles 714 drive the solid waste to above the solid waste tank 740. In the present example, the cylindrical drum is rotated in the second rotational direction R2 which causes the baffles 714 to drive to solid waste to the second longitudinal end 713B of the drum 710 and, therefore, above the solid waste tank 740. The solid waste may be considered disposed above the solid waste tank 740 after a predetermined number of revolutions of the drum.

When the solid waste is disposed above the solid waste tank 740, the sheathe member 750 is rotated relative to the drum 710 to expose the opening 791 in the drum 710 which results in the solid waste falling through the opening under gravity into the solid waste tank 740. As described herein, the longitudinal extent of the opening 191 is less than or equal to the longitudinal extent of the solid waste tank 740, which thereby prevents the solid waste missing the solid waste tank 740 when falling through the opening.

The solid waste tank 740 is removable. Example solid waste tanks are described in more detail herein.

The bioreactor apparatus may be configured to include any of a manual food waste loader, a caddy food waste loader and a bin food waste loader. These loaders are described in more detail herein.

A food waste input 190 such as that described in more detail herein may be used with the bioreactor 700.

The apparatus and methods described herein may permit food waste to be mixed with the recirculated slurry which contains microorganisms for breakdown (e.g. digestion) thereof. Fresh water can be added from the one or more sprayers. The mechanical action of the drum, including the rotation and the shearing and cutting of the baffles, will break up the food waste solids. The biological actions of the microorganisms will hydrolyse the cellulose fibres into smaller molecules (thus break up the fibres), convert organic matters, like carbohydrates (starch and sugars), proteins and lipids into soluble organic acids. This produces a homogeneous slurry with high concentration of both soluble organic compounds and fine organic particles.

Certain features of the methods described herein may be implemented in hardware, and one or more functions of the apparatus may be implemented in method steps. It will also be appreciated in the context of the present disclosure that the methods described herein need not be performed in the order in which they are described, nor necessarily in the order in which they are depicted in the drawings. Accordingly, aspects of the disclosure which are described with reference to products or apparatus are also intended to be implemented as methods and vice versa. The methods described herein may be implemented in computer programs, or in hardware or in any combination thereof. Computer programs include software, middleware, firmware, and any combination thereof. Such programs may be provided as signals or network messages and may be recorded on computer readable media such as tangible computer readable media which may store the computer programs in non-transitory form. Hardware includes computers, handheld devices, programmable processors, general purpose processors, application specific integrated circuits (ASICs), field programmable gate arrays (FPGAs), and arrays of logic gates.

Any processors used in the computer system (and any of the activities and apparatus outlined herein) may be implemented with fixed logic such as assemblies of logic gates or programmable logic such as software and/or computer program instructions executed by a processor. The computer system may comprise a central processing unit (CPU) and associated memory, connected to a graphics processing unit (GPU) and its associated memory. Other kinds of programmable logic include programmable processors, programmable digital logic (e.g., a field programmable gate array (FPGA), a tensor processing unit (TPU), an erasable programmable read only memory (EPROM), an electrically erasable programmable read only memory (EEPROM), an application specific integrated circuit (ASIC), or any other kind of digital logic, software, code, electronic instructions, flash memory, optical disks, CD-ROMs, DVD (RTM) ROMs, magnetic or optical cards, other types of machine-readable mediums suitable for storing electronic instructions, or any suitable combination thereof. Such data storage media may also provide the data store of the computer system (and any of the apparatus outlined herein).

It will be appreciated from the discussion above that the embodiments shown in the Figures are merely exemplary, and include features which may be generalised, removed or replaced as described herein and as set out in the claims. In the context of the present disclosure other examples and variations of the apparatus and methods described herein will be apparent to a person of skill in the art.

1. A bioreactor apparatus for converting food waste to a slurry, the apparatus comprising:
a drum, wherein the drum comprises:
a drum surface defining an interior volume, wherein the interior volume is configured to receive food waste;
a plurality of holes in the drum surface to permit fluid to enter and exit the interior volume; and
a central longitudinal axis, wherein the drum is configured to rotate about the central longitudinal axis; and
one or more sprayers configured to spray fluid at the drum to thereby permit fluid sprayed at the drum to enter the interior volume of the drum.

2. The bioreactor apparatus of claim 1, wherein the drum comprises one or more baffles on an interior of the drum surface, wherein the one or more baffles are configured to mechanically break solid parts of food waste disposed within the interior volume.

3. The bioreactor apparatus of claim 2, wherein each of one or more baffles comprises a plurality of teeth.

4. The bioreactor apparatus of claim 1, wherein the one or more sprayers are disposed over the drum.

5. The bioreactor apparatus of claim 4, comprising a slurry tank configured to collect fluid which exits from the drum, wherein the slurry tank is disposed under the drum.

6. The bioreactor apparatus of claim 5, wherein:
the drum comprises an openable drum port, wherein the openable drum port is capable of forming:
an open configuration wherein solid waste is discharged from the drum via the drum port; and,
a closed configuration wherein solid waste is not discharged from the drum via the drum port;
wherein the bioreactor apparatus further comprises a solid waste tank configured to collect solid waste discharged from the drum via the openable drum port.

7. The bioreactor apparatus of claim 6, wherein:
the slurry tank is disposed closer to the first longitudinal end than to the second longitudinal end;
the solid waste tank is disposed closer to the second longitudinal end than to the first longitudinal end; and
the drum port is disposed closer to the second longitudinal end than to the first longitudinal end and above at least a portion of the solid waste tank.

8. The bioreactor apparatus of claim 7, wherein the drum port comprises:
an opening in a portion of drum suitable for solid waste to pass therethrough; and
a sheathe member disposed around the exterior of at least a portion the drum;
wherein the sheathe member is either:
rotatable around the longitudinal axis and relative to the drum; or
movable along the longitudinal axis and relative to the drum;
wherein the drum port is in the closed configuration when the sheathe member covers the opening; and
wherein the drum port is in the open configuration when the sheathe member does not cover the opening.

9. The bioreactor apparatus of claim 7, wherein the drum comprises one or more baffles on an interior of the drum surface, wherein the one or more baffles are configured to mechanically break solid parts of food waste disposed within the interior volume, and wherein the one or more baffles are configured to:
move solid parts of food waste towards the first longitudinal end when the drum is rotated in a first rotational direction around the longitudinal axis; and
move solid parts of food waste towards the second longitudinal end when the drum is rotated in a second rotational direction around the longitudinal axis.

10. The bioreactor apparatus of claim 9, wherein rotating the drum in the second rotational direction switches the drum port from the closed configuration to the open configuration.

11. The bioreactor apparatus of claim 5, comprising a slurry recirculation system, configured to recirculate slurry from the slurry tank to the interior of the drum.

12. (canceled)

13. The bioreactor apparatus of claim 5, comprising:
an air-tight housing configured to enclose, within an interior of the housing, the drum, one or more sprayers, and the slurry tank,
wherein the air-tight housing is configured to prevent odorous gas exiting the interior of the housing.

14. The bioreactor apparatus of claim 13, comprising an openable food waste input, wherein the food waste input is capable of forming:
a first configuration wherein the food waste input is open to receive food waste and closes off the interior of the housing; and
a second configuration wherein the food waste input is closed to receive food and opens the food waste input to the interior of the housing to permit the food waste in the food waste input to enter the interior of the drum.

15. The bioreactor apparatus of claim 14, wherein:
the slurry tank is disposed closer to the first longitudinal end than to the second longitudinal end;
the solid waste tank is disposed closer to the second longitudinal end than to the first longitudinal end;
the drum port is disposed closer to the second longitudinal end than to the first longitudinal end and above at least a portion of the solid waste tank; and
the food waste input is disposed above the second longitudinal end of the drum to permit food to enter the interior of the drum via the drum port.

16. The bioreactor apparatus of claim 14, wherein the apparatus further comprises:
a caddy food waste loader configured to automatically input food waste in a food caddy into the food waste input; or
a bin food waste loader configured to automatically input food waste in a food bin into the food waste input.

17. A method of operating a bioreactor apparatus the method comprising:
rotating a drum around a longitudinal axis wherein the drum comprising a plurality of holes disposed through a radial surface thereof; and
spraying liquid from a one or more sprayers at the drum.

18. The method of claim 17, wherein rotating the drum comprises at least one of:
rotating the drum in a first direction around a longitudinal axis wherein the drum comprising a plurality of holes disposed through a radial surface thereof, to thereby move solid parts of food waste towards the first longitudinal end; and/or
rotating the drum in a second direction around the longitudinal axis to thereby move solid parts of food waste towards the second longitudinal end.

19. The method of claim 17 comprising introducing a culture of microorganisms in the drum wherein the microorganisms are configured to break down the food waste.

20. The method of claim 17 performed using a bioreactor apparatus for converting food waste to a slurry, the apparatus comprising:
a drum, wherein the drum comprises:
a drum surface defining an interior volume, wherein the interior volume is configured to receive food waste;
a plurality of holes in the drum surface to permit fluid to enter and exit the interior volume; and
a central longitudinal axis, wherein the drum is configured to rotate about the central longitudinal axis; and
one or more sprayers configured to spray fluid at the drum to thereby permit fluid sprayed at the drum to enter the interior volume of the drum.

21. A computer readable non-transitory storage medium comprising a program for a computer configured to cause a processor to perform the method of claim 17.
